(19) United States
(12) Patent Application Publication
(10) Pub. No.: US 2022/0347020 A1
(43) Pub. Date: Nov. 3, 2022

(54) ELASTIC DIAPER ELEMENT

(71) Applicant: RKW SE, Mannheim (DE)

(72) Inventors: Georg BALDAUF, Berlin (DE); Christoph WILLIG, Vreden (DE)

(73) Assignee: RKW SE, Mannheim (DE)

(21) Appl. No.: 17/784,798

(22) PCT Filed: Aug. 27, 2020
(86) PCT No.: PCT/EP2020/073933

§ 371 Date: Jun. 13, 2022

(30) Foreign Application Priority Data
Dec. 12, 2019 (DE) 10 2019 134 100.0

(51) Int. Cl. A61F 13/49; A61F 13/15

(52) U.S. Cl. CPC A61F 13/4902; A61F 13/15699

(57) ABSTRACT

An elastic diaper element is provided that includes an elastic layer (1) and at least one layer (2) made of a non-woven. The elastic element has connecting regions (5) between the elastic layer (1) and the non-woven layer (2). The connecting regions (5) extend in a preferred direction. The non-woven layer (2) is corrugated when the diaper element is in the unstretched condition in order to provide regions (6) as a reserve allowing stretching. The connecting regions (5) have interruptions (10) along their extent.

### TECHNICAL FIELD

The invention relates to a stretchable diaper element with an elastic layer and a layer made of nonwoven, wherein the elastic element has connecting regions between the elastic layer and the layer made of nonwoven and the connecting regions have an extent in a preferred direction, wherein the layer made of nonwoven is in corrugated form in the non-stretched state of the diaper element in order to ensure there are regions acting as reserve for enabling stretching.

In the case of diapers, elastic elements are used in order to ensure good fit and tightness. The diaper element according to the invention is preferably used as a diaper waistband. The material is likewise particularly suitable for the use as an elastic closure element, a so-called back ear on baby diapers.

The diaper element according to the invention comprises a layer made of an elastic material. The elastic layer is provided on at least one side with a layer made of a nonwoven fabric. Such nonwoven fabrics generally have a limited extensibility. In the case of the diaper waistband elements according to the invention, a nonwoven layer is therefore connected in a corrugated form to the elastic layer. The troughs are connected to the elastic layer, whereas the crests project away from the elastic layer and enclose a cavity toward the elastic layer. As a result of the corrugations, the nonwoven layer in the non-stretched state forms a reserve for enabling stretching of the element.

### BACKGROUND

DE 602 04 588 T2 describes a production method for an extensible elastic composite material. In this case, use is made of rolls which have a multiplicity of axially spaced-apart teeth which lie next to one another, which run in a circumferential direction and which have the same shape. The spacings between teeth lying next to one another form recessed grooves which run in the circumferential direction and which have the same configuration.

A diaper with an upper and a lower layer is described in DE 689 23 866 T2. An elastic band is fastened to the elastic layer in the non-loaded state. The elastic band is connected to the elastic layer over the whole area. After stretching and relaxation of the elastic band, shined regions are formed.

EP 217032 B1 relates to a laminate with an elastic material which, at points which are spaced apart from one another, is connected to at least one web which is to be laid in pleats. The elastic material is a non-corrugated, elastic fiber web.

A method for producing a corrugated stretch laminate is described in EP 1 807 035 B1. In this case, an elastic composition is applied in a molten state to a carrier web to form an elastic element. A stretch composite preform is formed by stretching the carrier web. The preform is then stretched. A substrate is connected to the stretched preform in order to form a corrugated stretch laminate upon relaxation of the stretched preform.

EP 2 024 178 B1 describes a method for producing an elastically stretchable laminate with three layers. The laminate comprises an elastic film and two layers made of non-elastic nonwoven fabric. In one variant, a nonwoven crepe fabric is used. A first elastic laminate is connected to a non-elastic nonwoven layer in a stretched state.

### SUMMARY

It is an object of the present invention to provide a diaper element which has favorable stretching properties and at the same time ensures high tear resistance. In the case of force loading, the element is intended to behave in a stretchable manner, but also to build up sufficient resistance, which provides the consumer with the feeling of a high-quality product. In the use as a “back ear”, the element must not tear since otherwise the diaper can no longer be closed. In this case, by way of a corrugated design, at least one nonwoven layer is intended to provide a sufficient but not excessive reserve for stretching of the element. The laminate is intended to pose no risk to health and be environmentally sustainable. In addition, the intention is for no odors to emanate from the product. Furthermore, the element is intended to have pleasant haptics. The diaper element is also intended to ensure optimum fit of the diaper on the body, such that cavities between the diaper and the body are avoided and leakage of liquid is prevented.

This object is achieved according to the invention by way of a diaper element, a method and the use including one or more of the features described herein. Preferred variants are provided in claims, the description, the exemplary embodiment and the drawing.

According to the invention, the connecting regions have interruptions in their extent. To produce the elastic laminate, use is preferably made of a method in which the corrugated nonwoven layer is impressed into the molten elastic layer with a roll which has elevations. According to the invention, use is made in this case of a roll whose elevations have interruptions.

Preferably, the interruptions amount to more than 5 percent by area, preferably more than 20 percent by area, in particular more than 30 percent by area, in relation to the total area of the connecting regions.

Furthermore, it has proven to be particularly favorable if the interruptions take up less than 90 percent by area, preferably less than 80 percent by area, in particular less than 70 percent by area, in relation to the total area of the connecting regions.

It has proven to be particularly favorable if connecting regions have a rectilinear alignment. In this case, the connecting regions may be configured in the form of rectilinear lines.

It has proven to be particularly favorable if the connecting regions have an extent perpendicular to the tensile direction of the diaper element.

Preferably, the connecting regions are embodied as interrupted lines. It has proven to be favorable if the spacing between these lines is more than 1 mm, preferably more than 2 mm, in particular more than 2.5 mm and/or less than 8 mm, preferably less than 7 mm, in particular less than 6 mm.

In one advantageous variant of the invention, segments of connecting regions alternate with segments of interruptions. This forms rows comprising segments of connecting regions and segments of interruptions that are offset directly with respect to one another.

In one variant of the invention, the corrugated profile of the nonwoven layer can be formed between two rolls which have elevations and indentations, wherein at least one of the two rolls is configured in the form of a ridged roll. The elevations of the one roll in this case engage in the indentations of the other roll, and vice versa. According to the invention, the elevations have interruptions.

In an alternative variant of the invention, the corrugated profile of the nonwoven layer is formed between a roll and a further element which, like the roll, also has elevations and indentations. The elevations of the roll in this case engage in the indentations of the element, and vice versa. The element extends in an arcuate manner as far as the extruded elastic layer, such that the corrugated profile of the nonwoven layer remains unchanged up to the connecting step.

The expression “nonwoven” relates to a substance which can be produced from continuous filaments and/or discontinuous fibers, without weaving or knitting, by means of processes such as spunbonding, carding or melt-blowing. The nonwoven fabric can comprise one or more layers of nonwoven, wherein each layer can contain continuous filaments or discontinuous fibers. Nonwoven can also comprise bicomponent fibers which can have fiber structures such as for example shell-core or side-by-side.

The expression “elastic” preferably relates to any material that, upon application of a directed force, is able to stretch to a stretched length of at least approximately 160% of its relaxed, original length without tearing or breaking and that, when the applied force is canceled, returns by at least approximately 55% of its extension, preferably substantially to its original length, that is to say the returned length is less than approximately 120%, preferably less than approximately 110%, more preferably less than approximately 105%, of the relaxed original length.

The expression “diaper” preferably relates to disposable absorption articles which absorb and trap fluids. The term encompasses, inter alia, diapers with closures, diaper pants, training pants, swim diapers, incontinence articles for adults and the like.

In the production of the diaper element, prior to the connecting step, the nonwoven layer is brought into a three-dimensional, corrugated shape by being guided via a special apparatus. This apparatus may be a roll which has elevations and, as a result, forms the corrugated profile of the nonwoven layer. In addition or as an alternative, prior to the connecting step, the nonwoven layer can be guided via an element which extends in an arcuate manner as far as the extruded elastic layer, such that the corrugated profile of the nonwoven layer remains unchanged up to the connecting step. The element may for example be configured in the form of a finger strip.

Besides the melting capacity of the extruder, the production of the laminate requires no energy from the outside, as would be the case for example in the event of ultrasonic welding. The melt solidifies and the connecting regions according to the invention form. The connecting regions are thus provided in such a way that the nonwoven layer is subjected to no thermal loading from the outside, as would be the case for example in the event of ultrasonic welding or melting of the elastic layer by means of heated rolls.

The connecting regions according to the invention are preferably configured in a strip-like manner in rows which are arranged next to one another. There is preferably a straight profile within a row. The alignment of the connecting regions is preferably perpendicular to the tensile direction of the diaper element, such that the individual rows are aligned transverse to the tensile direction of the diaper waistband.

The ratio of the elevated regions to the sunken regions of the apparatuses, which are used for forming the corrugated profile and during the connecting step, is important for an optimal diaper waistband laminate. This ratio is also referred to as the web-to-groove ratio. This web-to-groove ratio is preferably less than 1:1, preferably less than 1:2.

It has proven to be particularly expedient for the connecting regions to have a width of more than 0.1 mm, preferably more than 0.3 mm, in particular of more than 0.5 mm and/or a width of less than 1.5 mm, preferably less than 1.3 mm, in particular less than 0.9 mm.

In a preferred variant of the invention, the connecting regions have external zones in which no elastic material has penetrated. There is no form-fitting bond in the external zones of the connecting regions since these zones are free of elastic material. The external zones prevent a situation whereby elastic material pierces through the nonwoven layer.

There is a form-fitting bond of solidified elastic material and nonwoven material in the inner zones of the connecting regions. In this case, the nonwoven material is also preferably not incipiently melted in the inner zones, but rather the fibers are merely pushed into the elastic melt. The material of the elastic layer surrounds the filaments of the nonwoven layer, such that a form-fitting bond is produced in the inner zone of the connecting regions after solidification of the elastic layer.

In individual cases, it may also be the case that at least individual fibers of the nonwoven material are incipiently melted in the inner zone. It is also possible from a purely theoretical standpoint that, in the inner zone, the nonwoven material is in completely melted form in the elastic melt.

In all cases, after solidification of the elastic material, there is a form-fitting bond of nonwoven material and solidified material of the elastic layer in the inner zone.

Arranged between the connecting regions are regions in which the elevations of the corrugated nonwoven layer project away from the elastic layer in corrugated form and enclose cavities. These regions serve as reserve for the stretching of the diaper element, there being no connection between the nonwoven layer and the elastic layer in these regions.

With regard to the total area of the flat film, the reserve regions comprise a significantly greater proportion than the connecting regions. The proportion of the reserve regions is preferably more than 60%, in particular more than 70%, preferably more than 80%, of the total area. As total area, the surface of the solidified flat elastic layer is used as reference.

There is no connection of the nonwoven layer to the elastic layer in the reserve regions.

As a result of the corrugated design, the nonwoven layer of a laminate portion is significantly longer than the elastic layer. The nonwoven layer is preferably longer than the elastic layer by more than a factor of 1.5, in particular by more than a factor of 2.0, preferably by more than a factor of 2.5.

In a preferred variant of the invention, after the connecting operation, the laminate is stretched in a transverse direction. The stretching is preferably performed below the elongation at break of the nonwoven layer.

It has proven to be particularly expedient for the non-bonding regions, that is to say the reserve regions, to have a width of more than 1.5 mm, preferably more than 2 mm, in particular more than 2.5 mm and/or a width of less than 6 mm, preferably less than 5 mm, in particular less than 4 mm.

The bonding regions preferably have a width of more than 0.1 mm, in particular more than 0.2 mm, preferably more than 0.3 mm and/or a width of less than 1 mm, preferably less than 0.8 mm, preferably less than 0.6 mm.

The reserve regions or connecting regions are preferably configured in a strip-like manner transverse to the tensile direction of the diaper waistband.

In order to create the connecting regions, use is preferably made of rolls having a surface structure, wherein the height of the elevations is more than 100 μm, preferably more than 500 μm, in particular more than 1 mm and/or less than 8 mm, preferably less than 10 mm, in particular less than 12 mm.

The nonwoven layer is preferably composed of a hydroentangled nonwoven fabric. Hydroentanglement makes it possible to reorient the fibers in the nonwoven fabric, and therefore the original two-dimensional fiber alignment is transferred into a three-dimensional fiber orientation. The fibers are incorporated in the nonwoven to a more pronounced extent. This nonwoven layer preferably has a specific weight of 5 to 80 g/m², preferably of 10 to 70 g/m², in particular of 15 to 35 g/m².

The hydroentangled nonwoven fabric layer preferably involves nonwoven fabrics made of continuous filaments. On account of their production process, these fabrics provide a fibrous web which is preferably of loop-like configuration.

Spinnable polymers such as for example polyester, PLA, polyolefins, in particular polypropylene and polyethylene, can be used as material for producing the continuous filaments.

Particularly advantageous is the use according to the invention of hydroentangled nonwoven fabric as the corrugated nonwoven layer which forms reserve regions for stretching of the diaper element. As a result of the formation of corrugations, the hydroentangled nonwoven fabric deforms in such a way that the fibers in the connecting regions are stretched and, as a result, preferably undergo orientation. As a result, particularly advantageous connecting zones are provided, in which the molten material surrounds the stretched and aligned filaments of the hydroentangled nonwoven fabric and, after solidification, a particularly favorable form-fitting bond is produced. It has been determined, surprisingly, that a diaper element with particularly favorable properties is provided when a hydroentangled nonwoven fabric is used. The nonwoven layer made of the hydroentangled nonwoven fabric can be deformed in a particularly satisfactory manner. The nonwoven web supplied loses virtually no width during the production process in spite of the formation of corrugations.

The elastic layer preferably involves a polypropylene and/or polyethylene block copolymer. The elastic film preferably has a specific weight of 5 to 140 g/m², in particular of 10 to 130 g/m², preferably of 20 to 40 g/m².

As an alternative, the elastic layer can also be composed of an SBC (styrene block copolymer) or an elastic polyurethane.

In one variant of the invention, the elastic layer is of multi-layered construction and is preferably embodied as a co-extruded film. In an advantageous variant, said film comprises a “core layer” and a “skin layer” which is significantly thinner compared with said core layer. The skin layer preferably has a specific weight of less than 5 g/m², in particular less than 4 g/m², preferably less than 3 g/m² and/or more than 0.3 g/m², in particular more than 0.6 g/m², preferably more than 0.9 g/m².

In one variant, the core layer is embedded between two skin-layer outer layers.

The core layer is preferably composed of an elastic polyolefin or an SBC (styrene block copolymer) or a polyurethane.

The skin layer is preferably composed of a polyethylene, a polypropylene or an EVA (ethylene-vinyl acetate copolymer).

In an expedient variant of the invention, filled polyolefins are used as skin layer. Examples of fillers used are mineral materials such as calcium carbonate or talc. The filler proportion is preferably more than 60% by weight, in particular more than 70% by weight, preferably more than 80% by weight.

Blends can also be used to form the skin layer. In this case, blends of polyolefins with polystyrene and/or blends of polyolefins with PLA are suitable, for example. No filler is required in the case of such blends.

The skin layer is designed such that it ensures deblocking from the tacky core layer. In addition, the skin layer can be easily deformed and can be readily stretched.

In one variant of the invention, the diaper element comprises a nonwoven layer made of a carded nonwoven fabric. The carded nonwoven fabric used is preferably composed of polypropylene fibers and/or of mixtures of different fiber types, such as, for example, of polypropylene/viscose, polypropylene/polyamide, polypropylene/polyester, etc. The carded nonwoven fabric can also be composed of polypropylene and/or polyethylene copolymer. The specific weight per unit area of the carded nonwoven fabric is preferably 10 to 40 g/m², in particular between 15 to 25 g/m². The carded nonwoven fabric can be consolidated for example by means of a calender and/or by means of the action of air and/or water jet.

In the case of the diaper element, the elastic layer can also be embedded between two layers made of nonwoven. In this case, at least one layer is preferably composed of a hydroentangled nonwoven material. The second nonwoven layer can also be composed either of a hydroentangled nonwoven fabric or a carded nonwoven fabric or a spunbonded nonwoven fabric. The second nonwoven layer can also either be of corrugated form or have a flat profile.

By way of the design of the diaper element according to the invention, the diaper bears against the body in optimum fashion and ensures optimum fit. As a result of the height of its fold arrangement, the diaper element has a voluminous design and fills cavities between the diaper and the body, such that a leak is effectively prevented.

The laminate according to the invention preferably has a rectilinear pleating of the nonwoven surface and has a good rebound resilience. The transverse elasticity and plastic deformation after application of stretching, also referred to as set, of laminates pressed in the longitudinal direction with webs exhibits a proportional dependency with the bonding surface. In the case of the laminate according to the invention, the bonding surface is reduced. In this case, sufficient adhesion is retained at the same time. Preferably, the adhesion of the laminate according to the invention is more than 1 N/25 mm in the machine direction (MD) with simultaneous improvement in the elasticity in the cross direction (CD).

Both requirements are achieved with the reduction in the web pressing surface of the rolls used. In one variant of the invention, the webs of the rolls are provided with milled portions, that is to say broken through.

Preferably, use is made in this case of rolls with parallel webs and grooves. Rolls of identical extent which have webs and grooves and which are grooved in a corrugated manner have proven to be particularly advantageous. These preferably have an amplitude of approx. 1 to 6 mm at a frequency of approx. 30 to 100 mm. The rolls, which are grooved in a corrugated manner at a depth of approx. 0.5 to 10 mm, engage in one another and roll in opposite directions with identical extent and speed in an interengaging manner.

The use of rolls with parallel or corrugated webs has proven to be particularly favorable, wherein the webs have an intermittent height profile in the circumferential direction.

### BRIEF DESCRIPTION OF THE DRAWINGS

Further advantages and features of the invention emerge from the description of an exemplary embodiment with reference to drawings and from the drawings themselves.

In the drawings:

FIG. 1 shows a section through a diaper element according to the invention, and

FIG. 2 shows a schematic top view of the diaper element with a first variant of the arrangement of interruptions.

### DETAILED DESCRIPTION

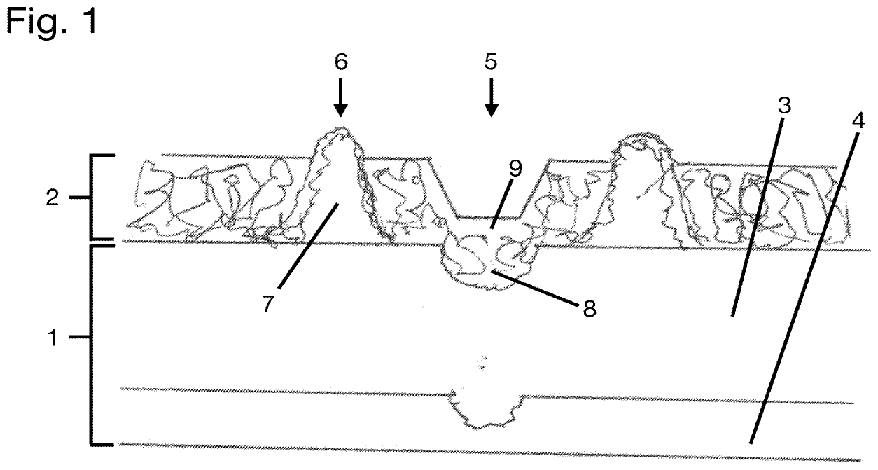

According to FIG. 1, the laminate comprises an elastic layer 1 and a nonwoven layer 2 made of a corrugated nonwoven fabric. This nonwoven fabric is a hydroentangled nonwoven fabric, a spunbonded nonwoven made of continuous filaments being used in the exemplary embodiment. According to the invention, the nonwoven fabric is brought into a corrugated form prior to the connection to the elastic layer 1. After the elastic layer 1 has been extruded onto the corrugated nonwoven layer 2, the indentations of the nonwoven fabric are pushed into the molten elastic layer 1, such that connecting regions 5 form between the corrugated nonwoven fabric 2 and the elastic layer 1.

In the exemplary embodiment, the elastic layer 1 is embodied as a multi-layered co-extruded film, having a core layer 3 and a further layer 4 which is configured in the form of a “skin layer”. The ratio of the thickness of the core layer 3 to the further layer 4 is preferably more than 8:1, in particular more than 10:1, in particular more than 12:1. The skin layer 4 preferably has a weight between 1 to 3 g/m².

The core layer 3 is preferably composed of thermoplastic polymers. In this case, preference is given to using polypropylene-polyethylene block copolymers, for example of the Exxon Vistamaxx (PP-based) product series: VM 6102 or VM 6202 or VM 7810 and/or of the Dow Infuse (PE-based) product series: Infuse 9507, Infuse 9107.

The outer layer 4 is preferably composed of a polyolefin or an ethylene-vinyl acetate copolymer (EVA). The outer layer 4, in contrast to the core layer 3, is not “tacky” and thus prevents undesired adhesion.

According to the invention, the laminate comprises connecting regions 5 and reserve regions 6. The reserve regions 6 exhibit no, or only a very weak, bond to the elastic layer 1 and preferably enclose cavities 7.

In the exemplary embodiment, the web-to-groove ratio of the roll which brings the nonwoven into a corrugated form and pushes the indentations of the corrugated nonwoven layer 2 into the molten elastic layer 1 is 1:6, past the web width is preferably 0.5 mm and the groove width is preferably 3 mm.

In the exemplary embodiment, the connecting regions 5 according to the invention have different zones 8, 9.

The external zone 9 is free of elastic material, such that the elastic material does not pierce through the nonwoven layer 2. The nonwoven material in the external zone 8 is not thermally influenced from the outside, such that the filaments of the layer 2 made of nonwoven are not incipiently melted.

There is a form-fitting bond of solidified elastic material and nonwoven material in the inner zone 8 of the connecting regions 5. In the exemplary embodiment, the nonwoven material is also not incipiently melted in the inner zone 8 in this case. The continuous filaments of the hydroentangled nonwoven fabric are merely pushed into the elastic melt, such that a form-fitting bond is produced after solidification. In this case, during the connecting process, the continuous filaments of the hydroentangled nonwoven fabric themselves remain largely uninfluenced. They are merely surrounded by the molten material of the elastic layer 1.

After solidification of the elastic material, there is a form-fitting bond of nonwoven material and solidified material of the elastic layer 1 in the inner zone 8.

The laminate illustrated in the figure is connected together between a pair of rolls, in the case of which, looking at the drawing, a profiled roll with elevations pushes the nonwoven layer 2 into the elastic layer 1 from above and, from below, there is arranged a mating roll with a smooth surface. In the production of the laminate illustrated in the figure, a chill roll is used as mating roll. The chill roll is a steel roll. The roll which acts from above is a non-cooled roll.

The rolls used for connection are driven at a spacing, wherein a fixed spacing is set.

The laminate preferably has a specific weight per unit area of more than 10 g/m², in particular more than 20 g/m², preferably more than 30 g/m² and/or less than 200 g/m², in particular less than 150 g/m², preferably less than 100 g/m².

In the exemplary embodiment, the connecting regions 5 and the reserve regions 6 are of strip-like design, wherein the strips run transversely with respect to the tensile direction of the diaper element.

The strips of the connecting regions 5 have a width of between 0.1 and 1 mm. In the exemplary embodiment, the connecting regions 5 have a width of 0.5 mm.

The strips of the reserve regions 6, in which there is no connection between the nonwoven layer 2 and the elastic layer 1, have a width of between 2 and 6 mm. In the exemplary embodiment, the reserve regions 6 have a width of 3 mm. In the exemplary embodiment, the connecting regions 5 thus have a proportion of 0.5/3.5=14.3%, and the reserve regions 6 have a proportion of 3/3.5=85.7%, in relation to the surface of the flat, non-stretched elastic layer 1.

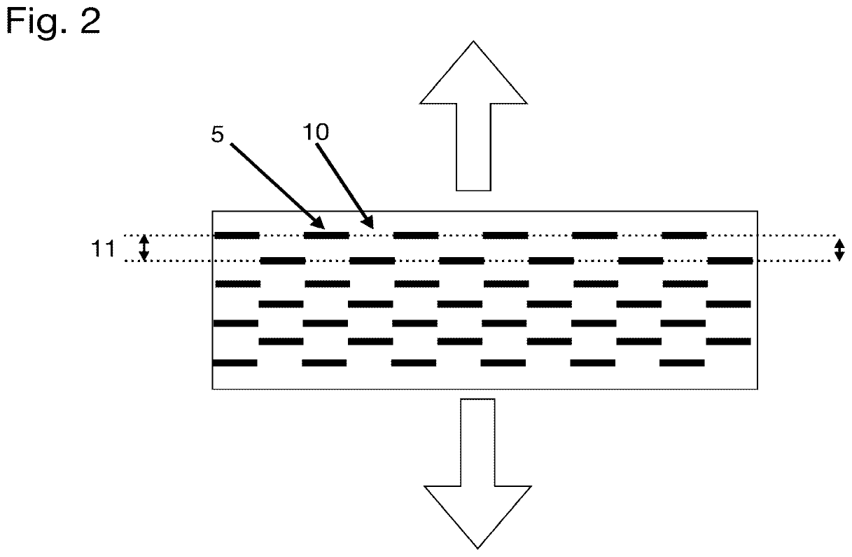

FIG. 2 shows schematic top view of the diaper element with a first variant of the arrangement of interruptions. The connecting regions 5 extend transversely with respect to the tensile direction and have, according to the invention, interruptions 10. The connecting regions 5 are embodied as interrupted lines, wherein the spacing 11 between these lines is approx. 3 mm in the exemplary embodiment. The block arrows which point upward and downward in the drawing show the tensile direction during loading of the diaper element.

1. A stretchable diaper element, comprising: an elastic layer;
a layer made of a nonwoven;
connecting regions between the elastic layer and the layer made of the nonwoven, the connecting regions have an extent in a preferred predetermined direction;
the layer made of the nonwoven is corrugated in a non-stretched state of the diaper element to provide reserve regions configured for enabling stretching; and
the connecting regions have interruptions in their extent.

2. The diaper element as claimed in claim 1, wherein the interruptions take up at least one of more than 5 percent by area or less than 90 percent by area in relation to a total area of the connecting regions.

3. The diaper element as claimed in claim 1, wherein the connecting regions have a rectilinear extent.

4. The diaper element as claimed in claim 1, wherein the connecting regions have an extent perpendicular to a tensile direction of the diaper element.

5. The diaper element as claimed in claim 1, wherein the connecting regions are configured as interrupted lines, and a spacing between the interrupted lines is at least one of more than 1 or less than 8.

6. The diaper element as claimed in claim 1, wherein the layer made of the nonwoven is composed of a hydroentangled nonwoven fabric, and the nonwoven fabric has a specific weight of 10 to 70 g/m².

7. The diaper element as claimed in claim 1, wherein the elastic layer is a multi-layered construction and comprises a core layer and at least one further layer.

8. The diaper element as claimed in claim 1, further comprising a further layer made of the nonwoven such that there are two layers made of the nonwoven, and the elastic layer is arranged between the two layers made of the nonwoven.

9. A method for producing an elastic laminate, comprising the following steps:
forming a corrugated layer made of a nonwoven,
extruding an elastic layer,
impressing the corrugated layer made of the nonwoven into the elastic layer while it is molten with a roll which has elevations, and
forming the elevations with interruptions.

10. An elastic laminate comprising a stretchable diaper element produced according to the method of claim 9.
